(19) United States
(12) Patent Application Publication
(10) Pub. No.: US 2005/0131041 A1
(43) Pub. Date: Jun. 16, 2005

(54) Azole derivatives as antifungal agents

(76) Inventors: Mohammad Salman, Plainsboro, NJ (US); Ashwani Kumar Verma, New Delhi (IN); Ashok Rattan, New Delhi (IN)

Correspondence Address: Jayadeep R Deshmukh;Ranbaxy Inc, 600 College Road East, Suite 2100, Princeton, NJ, 08540, US

(21) Appl. No.: 10/511,399

(22) PCT Filed: Apr. 17, 2002
(86) PCT No.: PCT/IB02/01242

(51) Int. Cl. A61K 31/4196; A61K 31/4178; C07D 43/02

(52) U.S. Cl. 514/383; 514/389; 514/397; 514/400; 548/266.2; 548/267.2; 548/312.7; 548/338.1

(57) ABSTRACT

The present invention relates to novel azole derivatives of Formula I, as potential antifungal agents. This invention also relates to pharmaceutical compositions containing the compounds of the present invention and their use in treating and/or preventing the fungal infections in mammals, preferably humans.

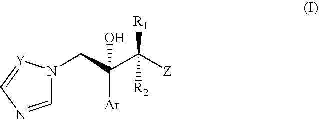

### FIELD OF THE INVENTION

The present invention relates to azole derivatives of Formula I, 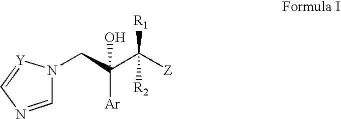
as potential antifungal agents.

This invention also relates to pharmaceutical compositions containing the compounds of the present invention and their use in treating and/or preventing the fungal infections in mammals, preferably humans.

### BACKGROUND OF THE INVENTION

Life threatening, systemic fungal infections continue to be a significant problem in health care. In particular, patients who become “immunocompromised” as a result of diabetes, cancer, prolonged steroid therapy, organ transplantation anti-rejection therapy, the acquired immune deficiency syndrome (AIDS) or other physiologically or immunologically compromising syndromes, are especially susceptible to opportunistic fungal infections.

Since the 1950s and until recently, the key opportunistic fungal pathogens were Candida albicans, Aspergillus fumigatus and Zygomycetes, which cause mucormycosis, a rapidly fatal infection especially in diabetic patients. Today, non-albicans Candida isolates have become more frequent, as have other Aspergillus species. Candida species are now the fourth most common cause of nosocomial blood stream infection and they are associated with an extremely high mortality rate of 40%. From 1980 to 1990, the incidence of fungal infections in the US hospitals nearly doubled from approximately 2 to 3.85 per 1000 patient days. The most marked increase in fungal infection rates occurred not only in transplant units or oncology centres, but also in surgical services. These changing patterns demonstrate that fungal infections are no longer limited to the most severely immunocompromised patients.

During the past two decades, a substantial shift in the epidemiology of candidemia due to different Candida species, has occurred. In the 1960s and 1970s Candida albicans accounted for 8590% of candidemia. In 1999, however, only 42% of candidemia cases were caused by C. albicans, while non-albicans Candida accounted for the remainder.

Cryptococosis is a leading cause of morbidity among the AIDS patients. The incidence of life threatening cryptococcal infection among these patients have been estimated to vary from 10 to 30%; 10-20% of the patients die during initial therapy and 30 to 60% patients succumb within a year. Penicillinium mameffei has been frequently isolated from HIV positive patients, especially in Southeast Asia.

The most common causative agent of mucormycosis is Rhizopus, a common bread mould that lives on any organic material. Other pathogens include Mucor, Rhizomucor and Absidia. Zygomycetes include twenty different fungi, all appearing the same histologically. The severely immunocompromised patient may become infected with Zygomycetes via respiratory inhalation.

Fusarium is the most prevalent plant fungus worldwide, and it is now recognised as a human pathogen as well. Fusarium infections can occur in immunocompetent or immunosuppressed individuals. Fusarium infection is life-threatening and associated with a poor prognosis.

Penicillium mameffei is an environmental fungi that can cause serious life-threatening infections in immunosuppressed patients. Penicillium mameffei has gained particular attention during the AIDS pandemic, as it may produce disease that is clinically indistinguishable from disseminated histoplasmosis.

Invasive aspergillosis has become a leading cause of death, mainly among patients suffering from acute leukaemia or after allogenic bone narrow transplant and after cytotoxic treatment of these conditions. It also occurs in patients with condition such as AIDS and chronic granulomatous disease. At present, only Amphotericin B and itraconazole are available for treatment of aspergillosis. In spite of their activity in vitro, the effect of these drugs in vivo against Aspergillus fumigatus remains low and as a consequence mortality from invasive aspergillosis remains high.

Although the first agent with antifungal activity, Griseofulvin was isolated in 1939 and the first azole and polyene antifungal agents were reported in 1944 and 1949, respectively (Clin. Microbiol, Rev., 1988; 1:187), it was not until 1960 that Amphotericin B (I.J. Am. Acad, Dermatol, 1994; 31:S51), which is still the “gold standard” for the treatment of severe systemic mycoses, was introduced (Antimicrob. Agents Chemother., 1996; 40:279). Despite the general effectiveness of Amphotericin B, it is associated with a number of complications and unique toxicities that limit its use. Furthermore, the drug is poorly absorbed from the gastrointestinal tract necessitating intravenous administration and also penetrates poorly into the cerebrospinal fluid (CSF) of both normal and inflamed meninges. The problems associated with Amphotericin B stimulated search for newer agents.

By 1980, members of the four major classes of antifungal agents, viz. polyenes, azoles, morpholines and allylamines had been identified. And advances made during the 1990's led to the addition of some new classes such as the Candins, and the Nikkomycins (Exp. Opin. Investig. Drugs, 1997; 6:129). However, with 15 different marketed drugs worldwide, (Drugs, 1997; 53:549) the azoles are currently the most widely used and studied class of antifungal agents.

Azole antifungal agents prevent the synthesis of ergosterol, a major component of fungal plasma membranes, by inhibiting the cytochrome P-450 dependent enzyme lanosterol demethylase (referred to as 14-α-sterol demethylase or P-450_{DM}). This enzyme also plays an important role in the cholesterol synthesis in mammals. When azoles are present in therapeutic concentrations, their antifungal efficacy is attributed to their greater affinity for fungal P-450_{DM} than for the mammalian enzyme (Curr. Opin. Chem. Biol., 1997; 1:176).

The azole antifungals currently in clinical use contain either two or three nitrogens in the azole ring and are thereby classified as imidazoles (e.g. ketoconazole, miconazole and clotrimazole) or triazoles (e.g. itraconazole and fluconazole), respectively. With the exception of Ketoconazole, use of the imidazoles is limited to the treatment of superficial mycoses, whereas the triazoles have a broad range of applications in the treatment of both superficial and systemic fungal infections. Another advantage of the triazoles is their greater affinity for fungal rather than mammalian cytochrome P-450 enzymes.

The use of Ketoconazole is severely restricted partly due to its poor toxicity and pharmacokinetic profile and also the fact that none of the opportunistic fungal infections like aspergillosis, candidemia and cryptococcosis are responsive to it (Antifungal Agents, pgs 401-410 In. G. L. Mandel, J. E. Bennett and R. Dolin (ed) Principles and practice of infectious diseases, 4^{th} ed. Churchill Livingstone, Inc. New York, N.Y.). Fluconazole is the current drug of choice for treatment of infectious caused by Candida species and C. neoformans. However, management of serious infectious due to Candida species are becoming increasingly problematic because of rising incidence of non-albicans species and the emergence non-albicans isolates resistant to both amphotericin B and the newer azoles. (Am. J. Med., 1996; 100:617). Also, fluconazole's spectrum suffers because it has only weak inhibitory activity against isolates of Aspergillus species. With regard to the prevention of invasive aspergillosis, a number of antifungal regimens have been suggested for neutropenic patients but only itraconazole has been considered for primary prophylaxis. However, its activity in the clinic remains mixed as it shows variable oral availability, low solubility and very high protein binding besides causing ovarian cancer in animals.

Voriconazole, the fluconazole analog launched recently by Pfizer exhibits 1.6 and 160 fold greater inhibition of ergosterol P⁴⁵⁰_{DM} in C. albicans and A. fumigatus lysates respectively, compared to fluconazole (Clin. Microbiol. Rev., 1999; 12:40). Voriconazole was designed to retain the parenteral and oral formulation advantage of fluconazole while extending its spectrum to moulds, insufficiently treated yeasts and less common fungal pathogens. But though oral bioavailability of voriconazole is high, there is saturable metabolism which results in a more than proportional increase in exposure with increased oral and I.V. doses. Inter-individual variability in voriconazole pharmacokinetics is high and concerns about its occular toxicity potentials remain to be resolved.

The development of some of the earlier compounds which included SCH 39304 (Genoconazole), TAK-187, SCH-42427 (Saperconazole), BAY R-8783 (Electrazole) and D-0870 had to be discontinued as a result of safety concerns.

ER-30346 (Ravuconazole), the fluconazole analog under development shows anti-aspergillus profile, at best only equal to that of itraconazole. Schering Plough compound SCH 56592 (Posaconazole) shows potent broad spectrum activity against primary opportunistic fungal pathogens including Candida spp., C. neoformans and Aspergillus spp. However, it has a pharmacokinetic profile similar to that of itraconazole and is not detectable in CSF, even when the serum drug concentration after several days of treatment are 25 to 100 times above the MIC for the most resistant C. neoformans. (Antimicrobial Agents and Chemother, 1996; 40:1910, 36^{th} interscience Conference on Antimicrobial agents and chemotherapy, September 1996, New Orleans Abst. Drugs of the Future, 1996; 21:20).

Caspofungin is the first member of a new class of antifungal drugs (echinocandins). It reduces the synthesis of β(1,3)D-glucan, an essential structural cell wall component of fungi. The cell wall is a component of fungal cells that is not found in mammalian cells and loss of cell wall glucan results in osmotic fragility of the fungal organism. The activity of the drug on the cell wall is accomplished indirectly by non competitive inhibition of a gene whose product is a cell membrane protein responsible for glucan synthesis. But caspofungin is not active against Cryptococcus neoformans and is available only for IV use.

Despite the therapeutic success of azole antifungals in the market, there remains a significant need for improved, broad spectrum, better tolerated, less toxic, safe at efficacious doses and more potent antifungal compounds with minimal potential for development of resistance among target fungi.

### SUMMARY OF THE INVENTION

The object of the present invention is to provide a compound having the structure of Formula I, 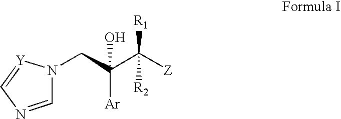
and its pharmaceutically acceptable salts, polymorphs, pharmaceutically acceptable solvates, enantiomers, diastereomers, N-oxides, prodrugs or metabolites, wherein:
Ar is a five to seven membered heterocyclic ring containing one to four heteroatoms selected from the group consisting of oxygen, nitrogen and sulphur; phenyl or a substituted phenyl having one to three substituents independently selected from halogen (e.g. chlorine, fluorine, bromine or iodine), nitro, cyano, lower(C₁₋₄)alkyl, lower(C₁₋₄)alkoxy, perhalo lower(C₁₋₄)alkyl or perhalo lower(C₁₋₄)alkoxy, the preferred heterocyclic rings are thienyl and pyridyl, the preferred Ar is halogen substituted phenyl and the more preferred halogen substituted phenyl is 2,4-difluorophenyl;
R₁ and R₂ are independently selected from the group consisting of hydrogen, straight chain or branched alkyl groups having 1 to 3 carbon atoms such as methyl, ethyl, propyl or isopropyl, the preferred alkyls are methyl and ethyl, the more preferred combination is when R₁ is methyl and R₂ is hydrogen,
   Y is CH or N;
   Z is selected from the group consisting of 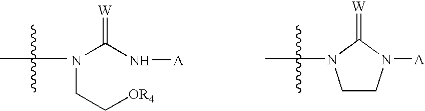
   wherein
W is selected from O, S, CH—NO₂ and N—CN;
A is hydrogen, unsubstituted or substituted lower(C₁₋₁₀)alkyl, the said substituents being halogen (e.g. fluorine, chlorine, bromine or iodine), hydroxy, lower(C₁₋₄)alkoxy, lower(C₁₋₄)perhaloalkyl, lower(C₁₋₄)perhaloalkoxy, unsubstituted or substituted C₆-C₁₀ aromatic or non aromatic with or without one to four heteroatoms selected independently from the group consisting of oxygen, nitrogen and sulphur, the said substituents can be independently selected from one or more groups such as halogen (e.g. fluorine, chlorine, bromine or iodine), nitro, cyano, hydroxy, lower(C₁₋₄)alkyl, lower(C₁₋₄)alkoxy, lower(C₁₋₄)perhaloalkyl, lower(C₁₋₄)perhaloalkoxy, BR₃, substituted or unsubstituted five or six membered heterocyclylic ring systems containing one to four heteroatoms selected from the group consisting of oxygen, nitrogen and sulphur, said heterocyclylic substituents being (C₁-C₈)alkanoyl, lower(C₁-C₄)alkyl, lower(C₁-C₄)alkoxy carbonyl, N lower(C₁-C₄)alkylaminocarbonyl, N,N-dilower(C₁-C₄)alkylaminocarbonyl, N-lower(C₁-C₄)alkylaminothiocarbonyl, N,N-di(lower alkyl)(C₁-C₄)aminothiocarbonyl, N-lower(C₁-C₄)alkyl sulphonyl, phenyl substituted lower(C₁-C₄)alkyl sulphonyl, N-lower(C₁-C₄)alkyl amino, N,N-di(lower alkyl)(C₁-C₄)amino, unsubstituted or substituted phenyl, the said substituents being halogen (e.g. fluorine, chlorine, bromine or iodine), hydroxy, lower(C₁₋₄)alkoxy, lower(C₁₋₄)perhaloalkyl, lower(C₁₋₄)perhaloalkoxy, niro, cyano, amino, N(R₄)₂, 5-6 membered heterocyclic rings the preferred heterocycles being 1,3-imidazolyl, 1,2,4 triazolyl; —CHR₅R₆.
   wherein
R₃ is five or six membered aromatic or non aromatic rings with or without heteroatoms (such as oxygen, nitrogen and sulphur);
B is independently selected from (CH₂)ₘ, —S, —O(CH₂)ₘ and —S(CH₂)ₘ;
m is an integer from 1 to 4;
R₄ is hydrogen, unsubstituted or substituted lower(C₁₋₄)alkyl;
R₅ is —COOR₄;
R₆ is independently selected from the group consisting of hydrogen, straight chain or branched alkyl with or without substituents, the said substituents being halogen (e.g. fluorine, chlorine, bromine or iodine), hydroxy, lower(C₁₋₄)alkyl, lower(C₁₋₄)alkoxy, lower(C₁₋₄)perhaloalkyl, lower(C₁₋₄)perhaloalkoxy, SR₄; phenyl or phenyl substituted with halogen (e.g. fluorine, chlorine, bromine or iodine), hydroxy, lower(C₁₋₄)alkoxy, lower(C₁₋₄)perhaloalkyl, lower(C₁₋₄)perhaloalkoxy, SR₄; heterocyclic rings or substituted heterocyclic rings with heteroatoms selected from oxygen, nitrogen and sulphur, substituents on heterocyclic rings are independently selected from halogen (e.g. fluorine, chlorine, bromine or iodine), hydroxy, lower(C₁₋₄)alkyl, lower(C₁₋₄)alkoxy, lower(C₁₋₄)perhaloalkyl, lower(C₁₋₄)perhaloalkoxy, SR₄; phenyl or phenyl substituted with halogen (e.g. fluorine, chlorine, bromine or iodine), hydroxy, lower(C₁₋₄)alkoxy, lower(C₁₋₄)perhaloalkyl, lower(C₁₋₄)perhaloalkoxy, SR₄ the preferred heterocyclic rings are imidazole and indole.

The present invention also provides pharmaceutical compositions for the treatment of fungal infections. These compositions comprise an effective amount of at least one of the above compounds of Formula I and/or an effective amount of at least one physiologically acceptable acid addition salts thereof with, a pharmaceutically acceptable carriers.

The compound represented by the Formula I may be used as its salt, examples of such salts are pharmacologically acceptable salts such as inorganic acid salts (e.g. hydrochloride, hydrobromide, sulphate, nitrate, phosphonate, etc.), organic acid salts (e.g. acetate, tartarate, citrate, fumarate, maleate, toluenesulphonate, and methanesulphonate, etc.). When carboxyl group is included in the Formula I as a substituent, it may be an alkali metal salt (e.g. sodium, potassium, calcium, magnesium and the like).

The present invention also includes within its scope, prodrugs of the compounds of Formula I. In general, such prodrugs will be functional derivatives of these compounds which are readily converted in vivo into defined compounds. Conventional procedures for the selection and preparation of suitable prodrugs are known.

The compounds represented by the formula I, or a salt thereof, have two or more stereoisomers due to the presence of one or more asymmetric centers atom in their molecule. It should be understood that any of such stereoisomers as well as a mixture thereof is within the scope of the present invention.

The invention also includes polymorphs and pharmaceutically acceptable solvates of these compounds, as well as metabolites. This invention further includes pharmaceutical compositions comprising the compounds of Formula I, their prodrugs, metabolites, enantiomers, diastereomers, N-oxides, polymorphs, solvates, or pharmaceutically acceptable salts thereof, in combination with a pharmaceutically acceptable carrier and optionally included excipients.

The illustrative list of particular compounds of the invention is given below:
1. 1-[(1R,2R)-2-(2,4-Difluorophenyl)-2-hydroxy-1-methyl-3-(1H-1,2,4-triazol-1-yl)propyl]-1-(2-hydroxyethyl)-3-[4-(1H-1-tetrazolyl)phenyl]thiourea.
2. 1-[(1R,2R)-2-(2,4-Difluorophenyl)-2-hydroxy-1-methyl-3-(1H-1,2,4-triazol-1-yl)propyl]-1-(2-hydroxyethyl)-3-[4-(2H-2-tetrazolyl)phenyl]thiourea.
3. 1-[(1R,2R)-2-(2,4-Difluorophenyl)-2-hydroxy-1-methyl-3-(1H-1,2,4-triazol-1-yl)propyl]-1-(2-hydroxyethyl)-3-[4-(2,2,3,3-tetrafluoropropoxy)phenyl]thiourea.
4. 1-[(1R,2R)-2-(2,4-Difluorophenyl)-2-hydroxy-1-methyl-3-(1H-1,2,4-triazol-1-yl)propyl]-3-[4-(1H-1-tetrazolyl)phenyl]-2-(1H,3H)-thioimidazolone.
5. 1-[(1R,2R)-2-(2,4-Difluorophenyl)-2-hydroxy-1-methyl-3-(1H-1,2,4-triazol-1-yl)propyl]-3-[4-(2H-2-tetrazolyl)phenyl]-2-(1H,3H)-thioimidazolone.
6. 1-[(1R,2R)-2-(2,4-Difluorophenyl)-2-hydroxy-1-methyl-3-(1H-1,2,4-triazol-1-yl)propyl]-3-[4-(2,2,3,3-tetrafluoropropoxy)phenyl]-2-(1H,3H)-thioimidazolone.
7. 1-[(1R,2R)-2-(2,4-Difluorophenyl)-2-hydroxy-1-methyl-3-(1H-1,2,4-triazol-1-yl)propyl]-3-[4-cyanophenyl]-2-(1H,3H)-thioimidazolone.
8. 1-[(1R,2R)-2-(2,4-Difluorophenyl)-2-hydroxy-1-methyl-3-(1H-1,2,4-triazol-1-yl)propyl]-3-[5-(2-chloropyridyl)]-2-(1H,3H)-thioimidazolone.

### DETAILED DESCRIPTION OF THE INVENTION

In order to achieve the above mentioned objectives and in accordance with the purpose of the invention as embodied and broadly described herein, there is provided a process for the synthesis of compound of Formula I, as shown in Schemes I and II. The starting materials for Scheme I and Scheme II may be suitably adapted to produce the more specific compounds of Formula I.

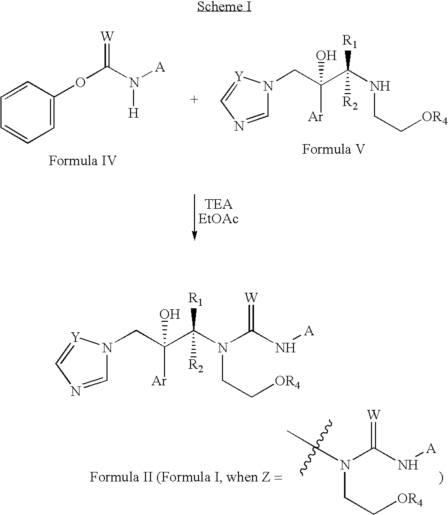

In Scheme 1, there is provided a process for preparing a compound of Formula II (Formula I, when 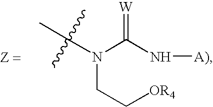
wherein
Ar is a five to seven membered heterocyclic ring containing one to four heteroatoms selected from the group consisting of oxygen, nitrogen and sulphur; phenyl or a substituted phenyl having one to three substituents independently selected from halogen (e.g. chlorine, fluorine, bromine or iodine), nitro, cyano, lower(C₁₋₄)alkyl, lower(C₁₋₄)alkoxy, perhalo lower(C₁₋₄)alkyl or perhalo lower(C₁₋₄)alkoxy;
R₁ and R₂ are independently selected from the group consisting of hydrogen, straight chain or branched alkyl groups having 1 to 3 carbon atoms including methyl, ethyl, propyl;
Y is CH or N;
W is selected from O, S, CH—NO₂ and N—CN;
A is hydrogen, unsubstituted or substituted lower(C₁₋₁₀)alkyl, the said substituents being halogen (e.g. fluorine, chlorine, bromine or iodine), hydroxy, lower(C₁₋₄)alkoxy, lower(C₁₋₄)perhaloalkyl, lower(C₁₋₄)perhaloalkoxy, unsubstituted or substituted C₁-C₁₀ aromatic or non aromatic rings with or without one to four heteroatoms independently selected from the group consisting of oxygen, nitrogen and sulphur, said substituents independently selected from one or more groups including halogen (e.g. fluorine, chlorine, bromine or iodine), nitro, cyano, hydroxy, lower(C₁₋₄)alkyl, lower(C₁₋₄)alkoxy, lower(C₁₋₄)perhaloalkyl, lower(C₁₋₄)perhaloalkoxy, BR₃; substituted or unsubstituted five or six membered heterocyclylic ring systems containing one to four heteroatoms are selected from the group consisting of oxygen, nitrogen and sulphur, said heterocyclylic substituents being (C₁-C₈)alkanoyl, lower(C₁-C₄)alkyl, lower(C₁-C₄)alkoxy carbonyl, N lower(C₁-C₄)alkylaminocarbonyl, N,N-dilower(C₁-C₄)alkylaminocarbonyl, N-lower(C₁-C₄)alkylaminothiocarbonyl, N,N-di(lower alkyl)(C₁-C₄)aminothiocarbonyl, N-lower(C₁-C₄)alkyl sulphonyl, phenyl substituted lower(C₁-C₄)alkyl sulphonyl, N-lower(C₁-C₄)alkyl amino, N,N-di(lower alkyl)(C₁-C₄)amino, unsubstituted or substituted phenyl, the said substituents being halogen (e.g. fluorine, chlorine, bromine or iodine), hydroxy, lower(C₁₋₄)alkoxy, lower(C₁₋₄)perhaloalkyl, lower(C₁₋₄)perhaloalkoxy, niro, cyano, amino, N(R₄)₂, 5-6 membered heterocyclic rings the preferred heterocycles being 1,3-imidazolyl, 1,2,4 triazolyl and —CHR₅R₆ wherein
R₃ is five or six membered aromatic or non aromatic rings with or without heteroatoms (including oxygen, nitrogen and sulphur);
B is independently selected from (CH₂)ₘ, —S, O(CH₂)ₘ and —S(CH₂)ₘ;
m is an integer from 1 to 4;
R₄ is hydrogen, unsubstituted or substituted lower(C₁₋₄)alkyl;
R₅ is —COOR₄;
R₆ is independently selected from the group consisting of hydrogen, straight chain or branched alkyl with or without substituents, the said substituents being halogen (e.g. fluorine, chlorine, bromine or iodine), hydroxy, lower(C₁₋₄)alkyl, lower(C₁₋₄)alkoxy, lower(C₁₋₄)perhaloalkyl, lower(C₁₋₄)perhaloalkoxy, SR₄; phenyl or phenyl substituted with halogen (e.g. fluorine, chlorine, bromine or iodine), hydroxy, lower(C₁₋₄)alkoxy, lower(C₁₋₄)perhaloalkyl, lower(C₁₋₄)perhaloalkoxy, SR₄, heterocyclic rings or substituted heterocyclic rings including imidazole and indole with heteroatoms selected from oxygen, nitrogen and sulphur, substituents on heterocyclic rings are independently selected from halogen (e.g. fluorine, chlorine, bromine or iodine), hydroxy, lower(C₁₋₄)alkyl lower(C₁₋₄)alkoxy, lower(C₁₋₄)perhaloalkyl, lower(C₁₋₄)perhaloalkoxy, SR₄; phenyl or phenyl substituted with halogen (e.g. fluorine, chlorine, bromine or iodine), hydroxy, lower(C₁₋₄)alkoxy, lower(C₁₋₄)perhaloalkyl, lower(C₁₋₄)perhaloalkoxy, SR₄; which comprises condensation of the compound of Formula IV with a compound of Formula V. The reaction of compounds of Formula IV and V is carried out in an organic solvent in the presence of a suitable base at a temperature ranging from 50-150° C., preferably at a temperature between 70-80° C.

The organic solvent is selected from the group consisting of ethyl acetate and N,N-dimethylformamide. The suitable base is selected from the group consisting of triethylamine, diisopropylamine, and pyridine.

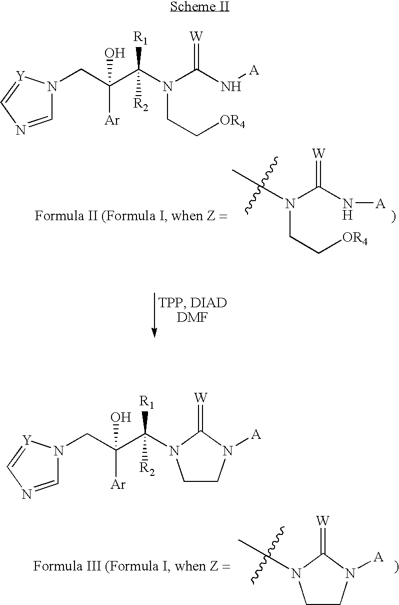

Scheme II shows the synthesis of compound of Formula III (Formula I, when 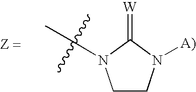
in which Ar, Y, R₁, R₂ and A have the same meanings as defined above, which comprises treating the compound of Formula II (Formula I, when Z 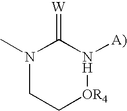
with triphenyl phosphine and diisopropyl azodicarboxylate (DIAD)/diethyl azodicarboxylate (DEAD) under Mitsunobu conditions to give the compound of Formula III.

The starting compound of Formula IV and Formula V of Scheme I can be prepared according to the process as described in U.S. Pat. No. 6,034,248 and Chem Pharm Bull., 2000; 48 (12):1947. The starting materials can be suitably adapted to produce the more specific compounds of Formula I.

In the above Schemes, where specific bases solvents, reagents etc. are mentioned, it is to be understood that other bases, reagents etc., known to those skilled in the art may also be used. Similarly, the reaction temperature and duration of the reactions may be adjusted according to the desired needs.

### Pharmacological Activity

The in vitro evaluation of the antifungal activity of the compounds of this invention (as shown in Table I) can be performed by determining the minimum inhibitory concentration (MIC) which is the concentration of the test compound in Rosewell Park Memorial Institute (RPMI) 1640 liquid medium buffered with 3-(Morpholino)propane sulfonic acid (MOPS) to pH 7, at which there is significant inhibition of the particular fungi. In practice the National Committee for Clinical Laboratory Standard (NCCLS) M27A document for Candida and Cryptococcus and M38P for Aspergillus was used to determine the MIC and readings recorded only when the Quality Control results fell into the acceptable range. After MIC results had been recorded, 20 μL from each of the well showing no growth was spotted on Sabouraud's Dextrose Agar (SDA) to determine the minimum fungicidal concentration (MFC).

To determine the in vivo efficacy of the compounds of this invention, lethal systemic infection models of infection in mice were established with Candida albicans, Cryptococcus neoformans and Aspergillus fumigatus. Mice, in groups of 6 per dose, were infected by the I.V. route by fungal spores at MLD concentration. Infected mice were randomised and dosed orally within 30 minutes of infection as appropriate. Mice were observed twice daily for 14 days at which time the experiment was terminated and ED₅₀ and/or MSD was calculated.

The in vivo evaluation of the compound can be carried out at a series of dose levels by oral or I.V. injection to mice which are inoculated I.V. with the minimum lethal dose of Candida albicans, Cryptococcus neoformans or Aspergillus fumigatus by the tail vein. Activity is based on the survival of a treated group of mice after the death of an untreated group of mice. For Aspergillus and Cryptococcus infections, target organs were cultured after treatment to document the number of mice cured of the infection for further assessment of activity.

For human use, the antifungal compound of the present invention and its salts can be administered as above, but will generally be administered in admixture with a pharmaceutical carrier selected with regard to the intended route of administration and standard pharmaecutical practice. For example, they can be administered orally in the form of tablets containing such excipients as starch or lactose or in capsules or ovules either alone or in admixture with excipients or in the form of elixirs, solutions or suspensions containing flavouring or colouring agents. They can be injected parenterally, for example, intravenously, intramuscularly or sub-cutaneously. For parenteral administration they are best used in the form of a sterile aqueous solution which may contain other substances, for example, enough salts or glucose to make the solution isotonic with blood.

TABLE 1

| | MIC (μg/ml) of standard drugs and Compounds of Present Invention | | | | | | |
|---|---|---|---|---|---|---|---|
| | | | | | Compound | Compound | Compound |
| Organism | FLU | AMB | ITRA | VORI | No. 4 | No. 5 | No. 6 |
| Candida parapsilosis 22019 | 2 | 0.125 | 0.03 | 0.03 | <0.00025 | <0.00025 | <0.00025 |
| (QC) | | | | | | | |
| Candida krusei 6258 (QC) | 32 | 0.25 | 0.125 | 0.25 | 0.25 | 0.06 | 0.125 |
| Paecilomyces variotti | 2 | 0.25 | 0.125 | 0.06 | 0.016 | Long trailing effect | |
| 22319(QC) | | | | | | | |
| Cryptococcus neoformans | 4 | 0.06 | 0.03 | 0.06 | 0.03 | <0.00025 | <0.00025 |
| M 106 | | | | | | | |
| Histoplasma capsulatum | 4 | 0.25 | 0.25 | 0.25 | 0.03 | 0.25 | 0.25 |
| Candida tropicalis 750 | 2 | 0.125 | 0.004 | 0.016 | 0.004 | <0.00025 | <0.00025 |
| Candida krusei 766.1 | 64 | 0.25 | 0.25 | 1 | 0.25 | 0.5 | 0.5 |
| Candida albicans Y-01-19 | 16 | 0.25 | 0.25 | 0.5 | 0.25 | 0.5 | 0.5 |
| Candida albicans 1122 | 0.5 | 0.25 | 0.016 | 0.16 | 0.06 | <0.00025 | <0.00025 |
| Candida glabrata 90030 | 16 | 0.5 | 0.5 | 1 | 0.06 | 1 | 2 |
| Aspergillus fumigatus 1008 | >128 | 0.25 | 0.25 | 0.25 | 0.25 | 0.125 | 0.125 |
| Aspergillus fumigatus Si-I | >128 | 0.5 | 0.125 | 0.25 | 0.25 | 0.016 | 0.016 |

FLU = FLUCONAZOLE
AMB = AMPHOTERICIN B
ITRA = ITRACONAZOLE
VORI = VORICONAZOLE

The invention is explained in detail in the examples given below which are provided by way of illustration only and therefore should not be constrained to limit the scope of the invention.

### EXAMPLE 1

### Preparation of 1-[(1R,2R)-2-(2,4-Difluorophenyl)-2-hydroxy-1-methyl-3-(1H-1,2,4-triazol-1-yl)propyl]-1-(2-hydroxyethyl)-3-[4-(1H-1-tetrazolyl)phenyl]thiourea

A mixture of 1-[(1R,2R)-2-(2,4-difluorophenyl)-2-hydroxy-1-methyl-3-(1H-1,2,3-trizol-1-yl)propyl]-1-(2-hydroxyethanol)(0.55 g), phenyl 4-(1H-1-tetrazolyl)phenyl thiocarbamate (0.75 g), triethylamine (0.205 g) and ethyl acetate (30 ml) was stirred under reflux for 15 h. After the reaction was over, the solvent was removed under reduced pressure and the residue was purified by column chromatography[silica gel 100-200 mesh; Dichloromethane: Ethyl acetate (9:1 to 1:9)] to afford the title compound (yield 0.6 g, 66%).

NMR(DMSO-d₆):—δ 10.36(s,1H; D₂O exchangeable), 10.07(s,1H), 8.25(s,1H), 7.88-7.85(d, 2H, 8.7 Hz), 7.665(m, 3H), 7.23(m, 2H), 6.96(q, 1H), 6.516(s, br, 1H; D₂O exchangeable), 6.18(s, 1H; D₂O exchangeable), 5.205-5.157(d, 1H, 14.5 Hz), 4.58-4.54(d, 1H, 14.5 Hz), 4.018(m, 4H) 0.966-0.856(d, 3H, 6.87 Hz) ppm.

The illustrative list of the compounds of the present invention prepared by the above method is given below
1-[(1R,2R)-2-(2,4-Difluorophenyl)-2-hydroxy-1-methyl-3-(1H-1,2,4-triazol-1-yl)propyl]-1-(2-hydroxyethyl)-3-[4-(2H-2-tetrazolyl)phenyl]thiourea.

NMR(CDCl₃):—δ 10.4(s, 1H), 8.66(s, 1H), 8.11-8.08(d, 2H, 8.7 Hz), 7.88(s, 1H), 7.706-7.67(d, 2H, 9.0 Hz), 6.767(m, 3H), 5.645-5.594(d, 1H, 15.3 Hz), 5.22(s, 1H; D₂O exchangeable), 4.385(m, 2H), 4.05(m, 2H0, 3.59(s, br, 1H; D₂O exchangeable) & 1.101-1078(d, 3H, 6.9 Hz) ppm.
1-[(1R,2R)-2-(2,4-Difluorophenyl)-2-hydroxy-1-methyl-3-(1H-1,2,4-triazol-1-yl)propyl]-1-(2-hydroxyethyl)-3-[4-(2,2,3,3-tetrafluoropropoxy)phenyl]thiourea.

NMR(DMSO-d₆):—δ 9.89(s, 1H; D₂O exchangeable), 8.22(s, 1H), 7.67(s, 1H), 7.24(m, 4H), 6.93(m, 3H), 6.69(tt, 1H; 41 Hz, 6.7 Hz), 6.537(q, 1H, 7.5 Hz), 6.16-6.11(d, 2H; 15 Hz), 5.21-5.16(d, 1H; 15 Hz), 4.56(q, 1H; 14 Hz), 3.996(m, 4H), & 0.947-0.924(d, 3H, 6.9 Hz) ppm.

### EXAMPLE 2

### Preparation of 1-[(1R,2R)-2-(2,4-Difluorophenyl)-2-hydroxy-1-methyl-3-(1H-1,2,4-triazol-1-yl)propyl]-3-[4-(1H-1-tetrazolyl)phenyl]-2-(1H, 3H)thiolmidazolone

A mixture of Compound No.1 (1.6 g) and triphenylphosphine (0.895 g) was dried under vacuum for 15 min. flushed with nitrogen and stirred in dimethylformamide (30 ml) at −5° C. followed by the addition of diisopropylazodicarboxylate (0.690 g) under nitrogen. The reaction mixture was then stirred at room temperature for 5 hr. After the reaction was over, it was poured into chilled water and extracted with ethyl acetate (3×100 ml). The combined organic layer was washed with water, dried over sodium sulphate and concentrated under reduced pressure to give foam which was purified by column chromatography [silica gel 100-200 mesh; Dichloromethane: Ethyl acetate (9:1 to 100% ethyl acetate) to give the title compound (yield 1.0 g, 64%).

NMR(CDCl₃):—δ 9.02(s, 1H), 7.85(m, 6H), 7.457(m, 1H), 6.826(m, 2H), 5.75(m, 1H), 5.417-5.369(d, 1H, 14.4 Hz), 5.29(s, 1H; D₂O exchangeable), 4.567-4.519(d, 1H, 14.4 Hz), 4.44(m, 2H), 4.14(m, 2H), 3.905(m, 1H) & 1.14-1.12 (d, 3H, 6.9 Hz) ppm.
1-[(1R,2R)-2-(2,4-Difluorophenyl)-2-hydroxy-1-methyl-3-(1H-1,2,4-triazol-1-yl)propyl]-3-[4-(2H-2-tetrazolyl)phenyl]-2-(1H, 3H)thioimidazolone.

NMR(CDCl₃):—δ 8.66(s, 1H), 8.21-8.18(d, 2H, 9.6 Hz), 7.84(m, 4H), 7.43(m, 1H), 6.79(m, 2H), 5.73(m, 1H), 5.41-5.36(d, 1H, 14.7 Hz), 5.256(s, 1H; D₂O exchangeable), 4.54-4.492(d, 1H, 14.4 Hz), 4.38(m, 1H), 4.107(m, 2H0, 3.869(m, 1H) & 1.11-1.08(d, 3H; 6.9 Hz) ppm.
1-[(1R,2R)-2-(2,4-Difluorophenyl)-2-hydroxy-1-methyl-3-(1H-1,2,4-triazol-1-yl)propyl]-3-[4-(2,2,3,3-tetrafluoropropoxy)phenyl]-2-(1H, 3H)thioimidazolone.

NMR(CDCl₃):—δ 7.85(s,1H), 7.78(s, 1H), 7.43(m, 3H), 6.908(m, 3H), 6.75(m, 2H) 6.04(tt, 1H; 55 Hz, 4.77 Hz), 5.65(q, 1H, 6.9 Hz), 5.358-5.31 (d, 1H; 14 Hz), 5.179(s, 1H; D₂O exchangeable), 4.522-4.47(d, 1H, 14.67 Hz), 4.30(m, 3H), & 1.057-1.30(d, 3H; 7 Hz) ppm.

While the present invention has been described in terms of its specific embodiments, certain modifications and equivalents will be apparent to those skilled in the art and are intended to be included within the scope of the present invention.

1. A compound having the structure of Formula I 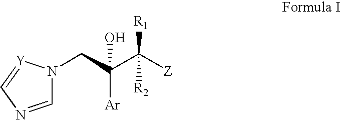
and its pharmaceutically acceptable salts, enantiomers, diastereomers, N-oxides, prodrugs, metabolites, polymorphs or pharmaceutically acceptable solvates,
wherein
Ar is a five to seven membered heterocyclic ring containing one to four heteroatoms selected from the group consisting of oxygen, nitrogen and sulphur; phenyl or a substituted phenyl having one to three substituents independently selected from halogen (e.g. chlorine, fluorine, bromine or iodine), nitro, cyano, lower(C₁₋₄)alkyl, lower(C₁₋₄)alkoxy, perhalo lower(C₁₋₄)alkyl or perhalo lower(C₁₋₄)alkoxy;
R₁ and R₂ are independently selected from the group consisting of hydrogen, straight chain or branched alkyl groups having 1 to 3 carbon atoms including methyl, ethyl, propyl;
Y is CH or N;
Z is selected from the group consisting of 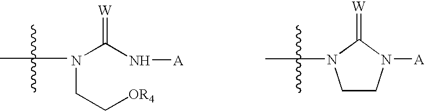
wherein
W is selected from O, S, CH—NO₂ and N—CN;
A is hydrogen, unsubstituted or substituted lower(C₁₋₁₀)alkyl, the said substituents being halogen (e.g. fluorine, chlorine, bromine or iodine), hydroxy, lower(C₁₋₄)alkoxy, lower(C₁₋₄)perhaloalkyl, lower(C₁₋₄)perhaloalkoxy, unsubstituted or substituted C₆-C₁₀ aromatic or non aromatic rings with or without one to four heteroatoms independently selected from the group consisting of oxygen, nitrogen and sulphur, the said substituents independently selected from one or more groups including halogen (e.g. fluorine, chlorine, bromine or iodine), nitro, cyano, hydroxy, lower(C₁₋₄)alkyl, lower(C-4)alkoxy, lower(C, 4)perhaloalkyl, lower(C₁₋₄)perhaloalkoxy, BR₃, substituted or unsubstituted five or six membered heterocyclylic ring systems containing one to four heteroatoms are selected from the group consisting of oxygen, nitrogen and sulphur, said heterocyclylic substituents being (C₁-C₈)alkanoyl, lower(C₁-C₄)alkyl, lower(C₁-C₄)alkoxy carbonyl, N lower(C₁-C₄)alkylaminocarbonyl, N,N-dilower(C₁-C₄)alkylaminocarbonyl, N-lower(C₁-C₄)alkylaminothiocarbonyl, N,N-di(lower alkyl)(C₁-C₄)aminothiocarbonyl, N-lower(C₁-C₄)alkyl sulphonyl, phenyl substituted lower(C₁-C₄)alkyl sulphonyl, N-lower(C₁-C₄)alkyl amino, N,N-di(lower alkyl)(C₁-C₄)amino, unsubstituted or substituted phenyl, the said substituents being halogen (e.g. fluorine, chlorine, bromine or iodine), hydroxy, lower(C₁₋₄)alkoxy, lower(C₁₋₄)perhaloalkyl, lower(C₁₋₄)perhaloalkoxy, nitro, cyano, amino, N(R₄)₂, 5-6 membered heterocyclic rings the preferred heterocycles being 1,3-imidazolyl, 1,2,4 triazolyl and —CHR₅R₆ wherein
R₃ is five or six membered aromatic or non aromatic rings with or without heteroatoms (including oxygen, nitrogen and sulphur);
B is independently selected from (CH₂)ₘ, —S, —O(CH₂)ₘ and —S(CH₂)ₘ;
m is an integer from 1 to 4;
R₄ is hydrogen, unsubstituted or substituted lower(C₁₋₄)alkyl;
R₅ is —COOR₄;
R₆ is independently selected from the group consisting of hydrogen, straight chain or branched alkyl with or without substituents, the said substituents being halogen (e.g. fluorine, chlorine, bromine or iodine), hydroxy, lower(C₁₋₄)alkyl, lower(C₁₋₄)alkoxy, lower(C₁₋₄)perhaloalkyl, lower(C₁₋₄)perhaloalkoxy, SR₄; phenyl or phenyl substituted with halogen (e.g. fluorine, chlorine, bromine or iodine), hydroxy, lower(C₁₋₄)alkoxy, lower(C₁₋₄)perhaloalkyl, lower(C₁₋₄)perhaloalkoxy, SR₄, heterocyclic rings or substituted heterocyclic rings including imidazole and indole with heteroatoms selected from oxygen, nitrogen and sulphur, substituents on heterocyclic rings are independently selected from halogen (e.g. fluorine, chlorine, bromine or iodine), hydroxy, lower(C₁₋₄)alkyl, lower (C₁₋₄)alkoxy, lower(C₁₋₄)perhaloalkyl, lower(C₁₋₄)perhaloalkoxy, SR₄; phenyl or phenyl substituted with halogen (e.g. fluorine, chlorine, bromine or iodine), hydroxy, lower(C₁₋₄)alkoxy, lower(C₁₋₄)perhaloalkyl, lower(C₁₋₄)perhaloalkoxy, SR₄.
The compound of claim 1 wherein Ar is thienyl, pyridyl, or halogen substituted phenyl.
The compound of claim 2 wherein Ar is 2,4-difluorophenyl.
The compound of claim 1 wherein R₁ and R₂ are independently selected from hydrogen, methyl and ethyl.
The compound of claim 1 wherein R, and R₂ are methyl and hydrogen, respectively.
A compound selected from the group consisting of:
1-[(1R,2R)-2-(2,4-Difluorophenyl)-2-hydroxy-1-methyl-3-(1H-1,2,4-triazol-1-yl)propyl]-1-(2-hydroxyethyl)-3-[4-(1H-1-tetrazolyl)phenyl]thiourea.
1-[(1R,2R)-2-(2,4-Difluorophenyl)-2-hydroxy-1-methyl-3-(1H-1,2,4-triazol-1-yl)propyl]-1-(2-hydroxyethyl)-3-[4-(2H-2-tetrazolyl)phenyl]thiourea.
1-[(1R,2R)-2-(2,4-Difluorophenyl)-2-hydroxy-1-methyl-3-(1H-1,2,4-triazol-1-yl)propyl]-1-(2-hydroxyethyl)-3-[4-(2,2,3,3-tetrafluoropropoxy)phenyl]thiourea.
1-[(1R,2R)-2-(2,4-Difluorophenyl)-2-hydroxy-1-methyl-3-(1H-1,2,4-triazol-1-yl)propyl]-3-[4-(1H-1-tetrazolyl)phenyl]-2-(1H, 3H)-thioimidazolone.
1-[(1R,2R)-2-(2,4-Difluorophenyl)-2-hydroxy-1-methyl-3-(1H-1,2,4-triazol-1-yl)propyl]-3-[4-(2H-2-tetrazolyl)phenyl]-2-thioimidazolone.
1-[(1R,2R)-2-(2,4-Difluorophenyl)-2-hydroxy-1-methyl-3-(1H-1,2,4-triazol-1-yl)propyl]-3-[4-(2,2,3,3-tetrafluoropropoxy)phenyl]-2-(1H, 3H)-thioimidazolone.
1-[(1R,2R)-2-(2,4-Difluorophenyl)-2-hydroxy-1-methyl-3-(1H-1,2,4-triazol-1-yl)propyl]-3-[4-cyanophenyl]-2-(1H,3H)-thioimidazolone.
1-[(1R,2R)-2-(2,4-Difluorophenyl)-2-hydroxy-1-methyl-3-(1H-1,2,4-triazol-1-yl)propyl]-3-[5-(2-chloropyridyl)]-2-(1H,3H)-thioimidazolone.
A pharmaceutical composition comprising the compound as defined in claims 1 or 6 and a pharmaceutically acceptable carrier or diluent.
A method of treating or preventing fungal infection in a mammal comprising administering to said mammal a therapeutically effective amount of a compound having the structure of Formula I 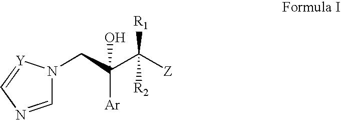
and its pharmaceutically acceptable salts, enantiomers, diastereomers, N-oxides, prodrugs, metabolites, polymorphs or pharmaceutically acceptable solvates,
wherein
Ar is a five to seven membered heterocyclic ring containing one to four heteroatoms selected from the group consisting of oxygen, nitrogen and sulphur; phenyl or a substituted phenyl having one to three substituents independently selected from halogen (e.g. chlorine, fluorine, bromine or iodine), nitro, cyano, lower(C₁₋₄)alkyl, lower(C₁₋₄)alkoxy, perhalo lower(C₁₋₄)alkyl or perhalo lower(C₁₋₄)alkoxy;
R₁ and R₂ are independently selected from the group consisting of hydrogen, straight chain or branched alkyl groups having 1 to 3 carbon atoms including methyl, ethyl, propyl;
Y is CH or N;
Z is selected from the group consisting of 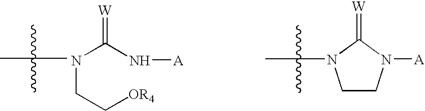
wherein
W is selected from O, S, CH—NO₂ and N—CN;
A is hydrogen, unsubstituted or substituted lower(C₁₋₁₀)alkyl, the said substituents being halogen (e.g. fluorine, chlorine, bromine or iodine), hydroxy, lower(C₁₋₄)alkoxy, lower(C₁₋₄)perhaloalkyl, lower(C₁₋₄)perhaloalkoxy, unsubstituted or substituted C₆-C₁₀ aromatic or non aromatic rings with or without one to four heteroatoms independently selected from the group consisting of oxygen, nitrogen and sulphur, the said substituents independently selected from one or more groups including halogen (e.g. fluorine, chlorine, bromine or iodine), nitro, cyano, hydroxy, lower(C₁₋₁₄)alkyl, lower(C₁₋₄)alkoxy, lower(C₁₋₄)perhaloalkyl, lower(C₁₋₄)perhaloalkoxy, BR₃, substituted or unsubstituted five or six membered heterocyclylic ring systems containing one to four heteroatoms are selected from the group consisting of oxygen, nitrogen and sulphur, said heterocyclylic substituents being (C₁-C₈)alkanoyl, lower(C₁-C₄)alkyl, lower(C₁-C₄)alkoxy carbonyl, N lower(C₁-C₄) alkylaminocarbonyl, N,N-dilower(C₁-C₄)alkylaminocarbonyl, N-lower(C₁-C₄)alkylaminothiocarbonyl, N,N-di(lower alkyl)(C₁-C₄)aminothiocarbonyl, N-lower(C₁-C₄)alkyl sulphonyl, phenyl substituted lower(C₁-C₄)alkyl sulphonyl, N-lower(C₁-C₄)alkyl amino, N,N-di(lower alkyl)(C₁-C₄)amino, unsubstituted or substituted phenyl, the said substituents being halogen (e.g. fluorine, chlorine, bromine or iodine), hydroxy, lower(C₁₋₄)alkoxy, lower(C₁₋₄)perhaloalkyl, lower(C₁₋₄)perhaloalkoxy, niro, cyano, amino, N(R₄)₂, 5-6 membered heterocyclic rings the preferred heterocycles being 1,3-imidazolyl, 1,2,4 triazolyl and —CHR₅R₆ wherein
R₃ is five or six membered aromatic or non aromatic rings with or without heteroatoms (including oxygen, nitrogen and sulphur);
B is independently selected from (CH₂)ₘ, —S, —O(CH₂)ₘ and —S(CH₂)ₘ;
m is an integer from 1 to 4;
R₄ is hydrogen, unsubstituted or substituted lower(C₁₋₄)alkyl;
R₅ is —COOR₄;
R₆ is independently selected from hydrogen, straight chain or branched alkyl with or without substituents, the said substituents being halogen (e.g. fluorine, chlorine, bromine or iodine), hydroxy, lower(C₁₋₄)alkyl, lower(C₁₋₄)alkoxy, lower(C₁₋₄)perhaloalkyl, lower(C₁₋₄)perhaloalkoxy, SR₄; phenyl or phenyl substituted with halogen (e.g. fluorine, chlorine, bromine or iodine), hydroxy, lower(C₁₋₄)alkoxy, lower(C₁₋₄)perhaloalkyl, lower(C₁₋₄)perhaloalkoxy, SR₄, heterocyclic rings or substituted heterocyclic rings including imidazole and indole with heteroatoms selected from oxygen, nitrogen and sulphur, substituents on heterocyclic rings are independently selected from halogen (e.g. fluorine, chlorine, bromine or iodine), hydroxy, lower(C₁₋₄)alkyl, lower(C₁₋₄)alkoxy, lower(C₁₋₄)perhaloalkyl, lower(C₁₋₄)perhaloalkoxy, SR₄; phenyl or phenyl substituted with halogen (e.g. fluorine, chlorine, bromine or iodine), hydroxy, lower(C₁₋₄)alkoxy, lower(C₁₋₄)perhaloalkyl, lower(C₁₋₄)perhaloalkoxy, SR₄.

9. A method of treating or preventing a fungal infection in a mammal comprising the step of administering to said mammal a therapeutically effective amount of the pharmaceutical composition according to claim 7.

10. A process for preparing a compound of Formula II (Formula I, when 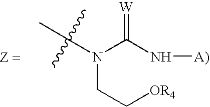 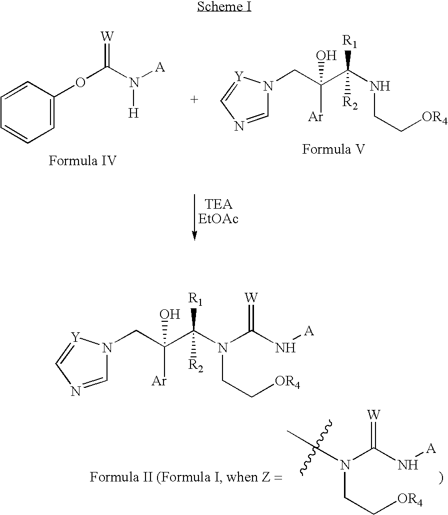
and its pharmaceutically acceptable salts, enantiomers, diastereomers, N-oxides, prodrugs, metabolites, polymorphs or pharmaceutically acceptable solvates,
wherein
Ar is a five to seven membered heterocyclic ring containing one to four heteroatoms selected from the group consisting of oxygen, nitrogen and sulphur; phenyl or a substituted phenyl having one to three substituents independently selected from halogen (e.g. chlorine, fluorine, bromine or iodine), nitro, cyano, lower(C₁₋₄)alkyl, lower(C₁₋₄)alkoxy, perhalo lower(C₁₋₄)alkyl or perhalo lower(C₁₋₄)alkoxy;
R₁ and R₂ are independently selected from the group consisting of hydrogen, straight chain or branched alkyl groups having 1 to 3 carbon atoms including methyl, ethyl, propyl;
Y is CH or N;
W is selected from O, S, CH—NO₂ and N—CN;
A is hydrogen, unsubstituted or substituted lower(C₁₋₁₀)alkyl, the said substituents being halogen (e.g. fluorine, chlorine, bromine or iodine), hydroxy, lower(C₁₋₄)alkoxy, lower(C₁₋₄)perhaloalkyl, lower(C₁₋₄)perhaloalkoxy, unsubstituted or substituted C₆-C₁₀ aromatic or non aromatic rings with or without one to four heteroatoms independently selected from the group consisting of oxygen, nitrogen and sulphur, the said substituents independently selected from one or more groups including halogen (e.g. fluorine, chlorine, bromine or iodine), nitro, cyano, hydroxy, lower(C₁₋₄)alkyl, lower(C₁₋₄)alkoxy, lower(C₁₋₄)perhaloalkyl, lower(C₁₋₄)perhaloalkoxy, BR₃, substituted or unsubstituted five or six membered heterocyclylic ring systems containing one to four heteroatoms are selected from the group consisting of oxygen, nitrogen and sulphur, said heterocyclylic substituents being (C₁-C₈)alkanoyl, lower(C₁-C₄)alkyl, lower(C₁-C₄)alkoxy carbonyl, N lower(C₁-C₄)alkylaminocarbonyl, N,N-dilower(C₁-C₄)alkylaminocarbonyl, N-lower(C₁-C₄)alkylaminothiocarbonyl, N,N-di(lower alkyl)(C₁-C₄)aminothiocarbonyl, N-lower(C₁-C₄)alkyl sulphonyl, phenyl substituted lower(C₁-C₄)alkyl sulphonyl, N-lower(C₁-C₄)alkyl amino, N,N-di(lower alkyl)(C₁-C₄)amino, unsubstituted or substituted phenyl, the said substituents being halogen (e.g. fluorine, chlorine, bromine or iodine), hydroxy, lower(C₁₋₄)alkoxy, lower(C₁₋₄)perhaloalkyl, lower(C₁₋₄)perhaloalkoxy, nitro, cyano, amino, N(R₄)₂, 5-6 membered heterocyclic rings the preferred heterocycles being 1,3-imidazolyl, 1,2,4 triazolyl and —CHR₅R₆ wherein
R₃ is five or six membered aromatic or non aromatic rings with or without heteroatoms (including oxygen, nitrogen and sulphur);
B is independently selected from (CH₂)ₘ, —S, —O(CH₂)ₘ and —S(CH₂)ₘ;
m is an integer from 1 to 4;
R₄ is hydrogen, unsubstituted or substituted lower(C₁₋₄)alkyl;
R₅ is —COOR₄;
R₆ is independently selected from hydrogen, straight chain or branched alkyl with or without substituents, the said substituents being halogen (e.g. fluorine, chlorine, bromine or iodine), hydroxy, lower(C₁₋₄)alkyl, lower(C₁₋₄)alkoxy, lower(C₁₋₄)perhaloalkyl, lower(C₁₋₄)perhaloalkoxy, SR₄; phenyl or phenyl substituted with halogen (e.g. fluorine, chlorine, bromine or iodine), hydroxy, lower(C₁₋₄)alkoxy, lower (C₁₋₄)perhaloalkyl, lower(C₁₋₄)perhaloalkoxy, SR₄, heterocyclic rings or substituted heterocyclic rings including imidazole and indole with heteroatoms selected from oxygen, nitrogen and sulphur, substituents on heterocyclic rings are independently selected from halogen (e.g. fluorine, chlorine, bromine or iodine), hydroxy, lower(C₁₋₄)alkyl lower(C₁₋₄)alkoxy, lower(C₁₋₄)perhaloalkyl, lower(C₁₋₄)perhaloalkoxy, SR₄; phenyl or phenyl substituted with halogen (e.g. fluorine, chlorine, bromine or iodine), hydroxy, lower(C₁₋₄)alkoxy, lower(C₁₋₄)perhaloalkyl, lower(C₁₋₄)perhaloalkoxy, SR₄;
which comprises condensation of the compound of Formula IV with a compound of Formula V, to give the desired compound of Formula II (Formula I, when

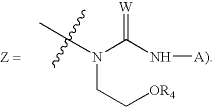

11. The process of claim 10 wherein Ar is thienyl, pyridyl, or halogen substituted phenyl.

12. The process of claim 11 wherein Ar is 2,4-difluorophenyl.

13. The process of claim 10 wherein R₁ and R₂ are independently selected from hydrogen, methyl and ethyl.

14. The process of claim 10 wherein R₁ and R₂ are methyl and hydrogen, respectively.

15. The process of claim 10 wherein the condensation of compound of Formula IV with a compound of Formula V is carried out in a suitable solvent selected from the group consisting of ethyl acetate and N,N-dimethylformamide.

16. The process of claim 10 wherein the condensation of compound of Formula IV with a compound of Formula V is carried out in the presence of a suitable base.

17. The process of claim 16 wherein the suitable base is selected from the group consisting of triethylamine, diisopropylamine and pyridine.

18. The process of claim 10 wherein the reaction is carried out at a temperature ranging from about 50-150° C.

19. A process for preparing a compound of Formula III (Formula I, when 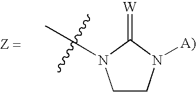 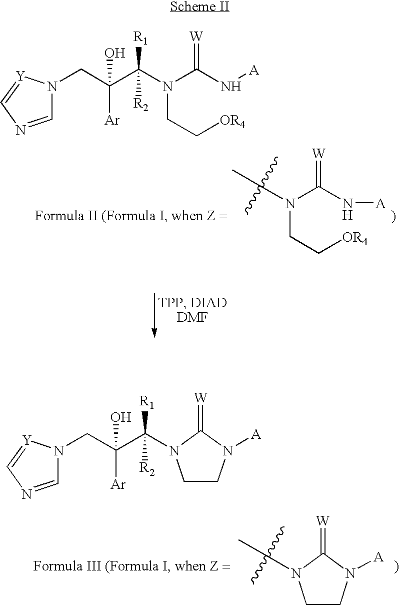
and its pharmaceutically acceptable salts, enantiomers, diastereomers, N-oxides, prodrugs, metabolites, polymorphs or pharmaceutically acceptable solvates thereof,
wherein
Ar is a five to seven membered heterocyclic ring containing one to four heteroatoms selected from the group consisting of oxygen, nitrogen and sulphur; phenyl or a substituted phenyl having one to three substituents independently selected from halogen (e.g. chlorine, fluorine, bromine or iodine), nitro, cyano, lower(C₁₋₄)alkyl, lower(C₁₋₄)alkoxy, perhalo lower(C₁)alkyl or perhalo lower(C₁₋₄)alkoxy;
R₁ and R₂ are independently selected from the group consisting of hydrogen, straight chain or branched alkyl groups having 1 to 3 carbon atoms including methyl, ethyl, propyl;
Y is CH or N;
W is selected from O, S, CH—NO₂ and N—CN;
A is hydrogen, unsubstituted or substituted lower(C₁₋₁₀)alkyl, the said substituents being halogen (e.g. fluorine, chlorine, bromine or iodine), hydroxy, lower(C₁₋₄)alkoxy, lower(C₁₋₄)perhaloalkyl, lower(C₁₋₄)perhaloalkoxy, unsubstituted or substituted C₆-C₁₀ aromatic or non aromatic rings with or without one to four heteroatoms independently selected from the group consisting of oxygen, nitrogen and sulphur, the said substituents independently selected from one or more groups including halogen (e.g. fluorine, chlorine, bromine or iodine), nitro, cyano, hydroxy, lower(C₁₋₄)alkyl, lower(C₁₋₄)alkoxy, lower(C₁₋₄)perhaloalkyl, lower(C₁₋₄)perhaloalkoxy, BR₃, substituted or unsubstituted five or six membered heterocyclylic ring systems containing one to four heteroatoms are selected from the group consisting of oxygen, nitrogen and sulphur, said heterocyclylic substituents being (C₁-C₈)alkanoyl, lower(C₁-C₄)alkyl, lower(C₁-C₄)alkoxy carbonyl, N lower(C₁-C₄)alkylaminocarbonyl, N,N-dilower(C₁-C₄)alkylaminocarbonyl, N-lower(C₁-C₄)alkylaminothiocarbonyl, N,N-di(lower alkyl)(C₁-C₄)aminothiocarbonyl, N-lower(C₁-C₄)alkyl sulphonyl, phenyl substituted lower(C₁-C₄)alkyl sulphonyl, N-lower(C₁-C₄)alkyl amino, N,N-di(lower alkyl)(C₁-C₄)amino, unsubstituted or substituted phenyl, the said substituents being halogen (e.g. fluorine, chlorine, bromine or iodine), hydroxy, lower(C₁₋₄)alkoxy, lower(C₁₋₄)perhaloalkyl, lower(C₁₋₄)perhaloalkoxy, niro, cyano, amino, N(R₄)₂, 5-6 membered heterocyclic rings the preferred heterocycles being 1,3-imidazolyl, 1,2,4 triazolyl and —CHR₅R₆ wherein
R₃ is five or six membered aromatic or non aromatic rings with or without heteroatoms (including oxygen, nitrogen and sulphur);
B is independently selected from (CH₂)ₘ, —S, —O(CH₂)ₘ and —S(CH₂)ₘ;
m is an integer from 1 to 4;
R₄ is hydrogen, unsubstituted or substituted lower(C₁₋₄)alkyl;
R₅ is —COOR₄;
R₆ is independently selected from hydrogen, straight chain or branched alkyl with or without substituents, the said substituents being halogen (e.g. fluorine, chlorine, bromine or iodine), hydroxy, lower(C₁₋₄)alkyl, lower(C₁₋₄)alkoxy, lower(C₁₋₄)perhaloalkyl, lower(C₁₋₄)perhaloalkoxy, SR₄; phenyl or phenyl substituted with halogen (e.g. fluorine, chlorine, bromine or iodine), hydroxy, lower(C₁₋₄)alkoxy, lower(C₁₋₄)perhaloalkyl, lower(C₁₋₄)perhaloalkoxy, SR₄, heterocyclic rings or substituted heterocyclic rings including imidazole and indole with heteroatoms selected from oxygen, nitrogen and sulphur, substituents on heterocyclic rings are independently selected from halogen (e.g. fluorine, chlorine, bromine or iodine), hydroxy, lower(C₁₋₄)alkyl, lower(C₁₋₄)alkoxy, lower(C₁₋₄)perhaloalkyl, lower(C₁₋₄)perhaloalkoxy, SR₄; phenyl or phenyl substituted with halogen (e.g. fluorine, chlorine, bromine or iodine), hydroxy, lower(C₁₋₄)alkoxy, lower(C₁₋₄)perhaloalkyl, lower(C₁₋₄)perhaloalkoxy, SR₄;
which comprises reacting the compound of Formula II under Mitsunobu reaction to give the compound of Formula III (Formula I, when

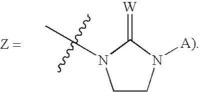

20. The process of claim 19 wherein the Mitsunobu reaction is carried out with triphenyl phosphine and diisopropyl azodicarboxylate (DIAD)/diethyl azodicarboxylate (DEAD).
